(19) United States
(12) Patent Application Publication
(10) Pub. No.: US 2009/0215146 A1
(43) Pub. Date: Aug. 27, 2009

(54) Method for Producing Virus-Type Particles Containing an Active Substance

(75) Inventors: Christoph Reichel, Martinsried (DE); Claus Rühland, Berlin (DE); Jurgen Hess, Baiersdorf (DE); Cristian Reiser, Bamberg (DE)

Correspondence Address: FISH & RICHARDSON P.C., P.O. BOX 1022, MINNEAPOLIS, MN, 55440-1022, US

(73) Assignee: responsif GmbH, Erlangen (DE)

(21) Appl. No.: 11/915,475

(22) PCT Filed: Mar. 28, 2006
(86) PCT No.: PCT/EP2006/002809

§ 371 Date: Dec. 11, 2008

(30) Foreign Application Priority Data
May 24, 2005 (DE) 102005024421.1

(51) Int. Cl. C12N 7/00; C12P 21/00

(52) U.S. Cl. 435/235.1; 435/69.7; 435/69.3

(57) ABSTRACT

The invention relates to a method for producing virus-type particles containing an active substance. Proteins, which comprise a first amino acid sequence which is derived from a first virus protein, and fusion proteins are assembled in order to form the virus-type particles. The proteins and the fusion proteins are coexpressed in yeast cells.

The invention relates to a method for producing virus-type particles containing an active substance as well as the virus-type particles which are produced by means of the method.

A synthetic biologically active molecule for anchoring an active substance to pentamers of the virus protein 1 (VP1) of the polyoma virus is known from the WO 00/61616 A1. Here, an amino acid sequence which binds to VP1 pentamers and is derived from the C terminal end of the virus protein 2 (VP2) and/or 3 (VP3) of the polyoma virus is bound at its one end to the active substance.

From the WO 99/25837 it is known to produce virus-type particles (Virus-Like Particles, VLPs) in yeasts by the expression of virus protein 1 of the polyoma virus (VP1).

From Palková, Z. et al., FEBS Letters 478 (2000), pages 281 to 289, it is known that the expression of the virus protein 1 of the polyoma virus in yeast greatly inhibits the growth of the yeast cells.

From Buonomassa, D. T. et al., Virology 293 (2002), pages 335 to 344, the co-expression of the human papillioma virus capsid proteins L1 and L2 and the assembling of these proteins to VLPs in yeasts are known.

From Kiessling, R., Proceedings of the Sixth Annual Walker's Cay Colloquium on Cancer Vaccines and Immunotherapy (2004), Keynote Address, page 4, it is known to express parts of the tumor antigen Her2/new at the carboxy terminal end of the VP2 structure capsid protein and to use polyoma VLPs in order to administer the Her2/new-VP2-construct to transgenic mice as tumor vaccines.

From Abbing, A. et al., Journal of Biological Chemistry (2004), volume 279, No. 26, pages 27410 to 27421, it is known to express both VP1 and a fusion protein which has the amino acid sequences of GFP and of the virus protein 2 of the polyoma virus (VP2), in E. coli. An assembling of VP1 and of the GFP-VP2 fusion protein occurs after their isolation in vitro. For this purpose, the proteins to be assembled are presented in a solution of an increased concentration and are incubated until VLPs are formed.

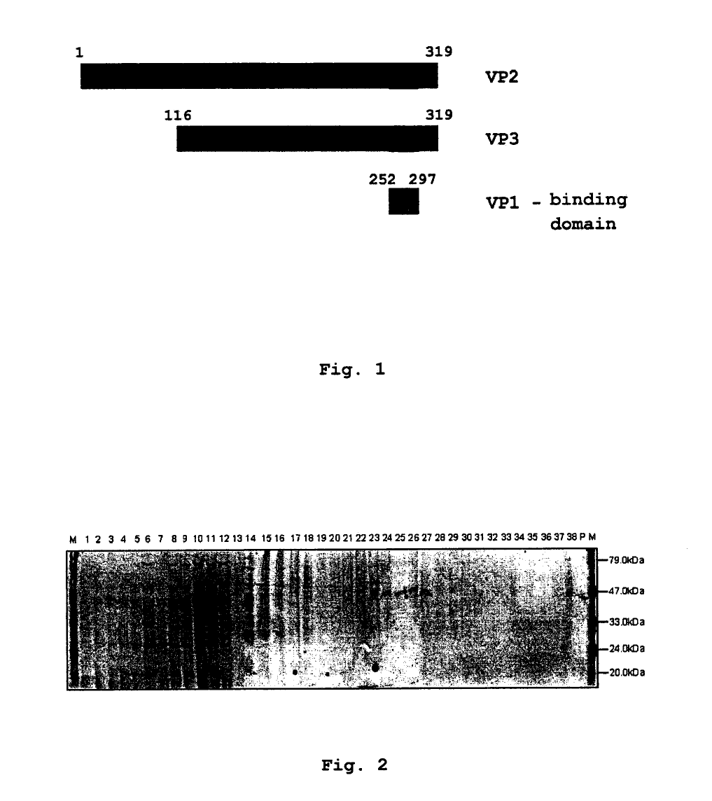

A fundamental problem in the expression of VP2 is that VP2 and in particular its domain situate at the C terminal end which is binding to VP1 pentamers (see FIG. 1) is comparatively hydrophobic and precipitates during an expression in E. coli already in a low concentration. Such a hydrophobic domain of VP2 produced in E. coli is often not able to specifically bind to VP1 pentamers. The expression in E. coli of recombinant VP2 which is specifically binding to VP1 pentamers may, however, be promoted by the expression of the amino acid sequence of VP2 together with a hydrophilic amino acid sequence—as with Abbing et al.—the amino acid sequence of GFP, as fusion protein.

During the expression of a recombinant fusion protein which comprises, apart from the domain of VP2 which interacts with a VP1 pentamer, a predominantly hydrophobic amino acid sequence, the problem consists in the correct protein folding. Since the domain of VP2 which usually interacts with VP1 pentamers is also hydrophobic, it can interact with the predominantly hydrophobic amino acid sequence thus exercising such a negative influence on the protein folding that the resulting fusion protein cannot specifically bind to VP1 pentamers. When such fusion proteins are expressed in E. coli, bodies are frequently formed which are also called “Inclusion Bodies”. Proteins which are contained in the inclusion bodies are denaturated and are not functional.

From Chen, S. X. et al., The EMBO Journal (1998), volume 17, No. 12, pages 3233 to 3240, a co-expression of VP1 and VP2 and/or VP3 in E. coli is known. Furthermore, it is known therefrom how the polyoma virus protein VP2 and/or VP3 interacts with the polyoma virus protein VP1 and which domains of the proteins are in each case responsible for this.

Many known tumor antigens contain strongly hydrophobic protein domains. These tumor antigens or peptides which contain sequences of the tumor antigen, including these protein domains, are frequently not soluble in water. For an immunisation of tumor patients with these tumor antigens, the same are usually dissolved in dimethylsulphoxide (DMSO) for their administration. This is for instance known from Gnjatic et al., Proc. Natl. Acad. Sci. USA (2002), volume 99, No. 18, pages 11813 to 11818. On account of possible side effects, such as skin reactions, disorders of the central nervous system, organic lesions to liver and kidneys, DMSO is considered to be harmful. Moreover, a dissolution of the tumor antigens and/or peptides in DMSO frequently does not bring about a structure as is present in the native tumor antigens. An immunisation that is effective against the native tumor antigens cannot be ensured thereby.

It is a characteristic feature of diseases with a chronic course that there exists a tolerance of the immunological system against specific disease-associated antigens. It is therefore a precondition for a successful immunotherapy that this tolerance is overcome. For this purpose, the induction of a B cell reaction is required which causes a specific antibody reaction against the antigens which are associated with the chronic disease.

In order to provide an effective immunological therapy for a chronic disease, such as a tumor, a chronic viral disease, as for instance an infection with HIV or HCV, or for another infectious disease, such as malaria, tuberculosis or bilharziosis, it is necessary that cytotoxic T cells are formed which are directed against tumor cells or against infected cells. This requires a correct and strong activation of the T cells. In this respect it is not sufficient to apply a tumor antigen or an antigen that is specific for the infected cells together with an auxiliary agent. It has been found out that what matters is a frequent repetition of the antigen motive in a small spatial interval and a good association of the antigen with an agent that stimulates the immuno-reaction, such as for instance VLPs. Therefore, the company Cytos Biotechnology AG, Switzerland, couples antigens in a covalent manner to the outside of recombinantly produced VLPs. This method has the disadvantage that it is time- and money-consuming and requires for an in vivo administration an additional cleaning in order to remove the reagents which are required for the covalent coupling.

It is an object of the present invention to eliminate the disadvantages of the state of the art. In particular a method is to be provided which permits the production of virus-type particles containing an active substance with the active substance to be presented in such a manner that it may trigger a formation of specific cytotoxic T cells in a mammal or in a human being. Furthermore, the virus-type particles and a use of the particles are to be stated.

According to the invention, the object is achieved by means of the features of Claims 1, 17 and 28. Opportune configurations of the invention follow from the features of Claims 2 to 16 and 18 to 27.

In accordance with the invention, a method for producing virus-type particles containing an active substance is proposed with proteins which each have a first amino acid sequence derived from a first virus protein and fusion proteins assembling to the virus-type particles. Here, the first amino acid sequence is an amino acid sequence which is adequate for the formation of capsoid-forming capsomers specifically binding a second virus protein. The fusion proteins have a second amino acid sequence each derived from the second virus protein and specifically binding to one of the capsomers each and a third amino acid sequence which forms the active substance. The proteins and the fusion proteins are co-expressed in yeast cells.

An amino acid sequence is derived from a protein when it is unchanged when compared to the complete or incomplete amino acid sequence of the protein or when it differs from it through amino acid exchanges, insertions or deletions.

By means of the co-expression of the fusion proteins in the yeast cells it is accomplished that the second amino acid sequence is folded in such a way that it may specifically bind to the capsomers even when the third amino acid sequence which forms the active substance is predominantly hydrophobic. In this way, virus-type particles containing the active substance may form from the fusion proteins and the capsomers formed from the proteins. Here, the active substance is arranged in such a way that it repeats frequently in a small spatial interval. A good association of the active substance with the virus-type particles is ensured by the specific binding to the capsomers of the domain of the fusion protein formed by the second amino acid sequence.

The particular feature of the method is that the VLPs produced in accordance with the method of the invention induce in mammals a strong immuno-reaction which is directed against the active substance. In this process, in particular also cytotoxic T cells are formed which attack cells that carry the active substance on their surface. Since the VLPs themselves exhibit the effect of an auxiliary agent, i. e. induce an immuno-reaction that is directed against the active substance, the administration of an additional auxiliary agent is not required. Compared to the method known from the company Cytos Biotechnology AG, no time- and money-consuming chemical binding of the active substance to the outside of the VLPs and no subsequent cleaning of the VLPs are required. Moreover, with the method of the invention, the active substance is positioned on the inside of the VLPs and is thus being protected from a degradation, for instance by proteases.

In the course of the co-expression of the proteins and of the fusion proteins in the yeast cells, at first capsomers are formed in the yeast cells from the proteins which each have the first amino acid sequence. From the capsomers and the fusion proteins VLPs form in the yeast cells which can then be isolated from the yeast cells.

Contrary to the co-expression in E. Coli known from Chen et al., the co-expression in yeasts does not entail the risk that, when isolating the VLPs from the cells used for co-expression, contamination by endotoxins is not completely separated from the VLPs. Thus, the expenditure for the cleaning of the VLPs which contain the active substance and for expensive toxicologic tests is distinctly reduced. When the cleaned VLPs produced in yeasts are administered to a mammal, the risk of an induction of fever or of an anaphylactic shock by endotoxin from the cells used for co-expression does not exist. In contrast to an expression in mammal cells, the risk of a contamination with human pathogenic agents, such as viruses, bacteria or prions does, moreover, not exist in the case of a co-expression in yeast cells.

Another advantage of the method according to the invention is that all proteins which are contained in the VLPs are produced and assembled in one organism. Consequently, it is not necessary, when a medicament is produced, to furnish a separate proof of the manufacture according to the GMP practice for all components. A single proof for the VLPs which are produced in yeast suffices.

Since the VLPs in their entirety are hydrophilic, they can be administered without any problem in an aqueous solution. The use of DMSO for administration is not required.

It is of particular advantage when the first virus protein and/or the second virus protein stems/stem from a virus or can be obtained from the same, the virus being selected from the group of non-enveloped viruses, comprising Papovaviridae, in particular Polyoma and Papilloma viruses, Iridoviridae, Adenoviridae, Parvoviridae, Picornaviridae, in particular polio viruses, Caliciviridae, Reoviridae and Birnaviridae. Preferably the first virus protein is the virus protein 1 of the polyoma virus (VP1), and the second virus protein is the virus protein 2 (VP2) or the virus protein 3 (VP3) of the polyoma virus. Preferably the capsomers have the form of pentamers, hexamers or heptamers.

The method according to the invention is of particular advantage when the third amino acid sequence is, at least predominantly, hydrophobic. The hydrophobicity of the amino acid sequence can for instance be ascertained by means of the method known from Kyte, J. and Russell, F. D., Journal of Molecular Biology (1982), Volume 157, edition 1, pages 105 to 132. Here, the hydrophilic and the hydrophobic properties of each of the 20 amino acid side chains are taken into account. An amino acid sequence is predominantly hydrophobic when the majority of the amino acids which constitute the sequence is hydrophobic or when a peptide/protein which has the amino acid sequence would be hardly soluble or insoluble in water.

The particularity of the co-expression of the proteins and of the fusion proteins in yeast cells is that per se hardly soluble or insoluble fusion proteins when they are formed interact immediately in situ with capsomers which have been formed from the proteins. This results in the formation of soluble complexes which then assemble to VLPs.

In contrast to the assembling of VP1 and VP2 fusion protein isolated from E. coli which is known from Abbing et al., the method according to the invention makes it possible for the first time to produce virus-type particles from capsomers and fusion proteins, with the fusion proteins each comprising a predominantly hydrophobic amino acid sequence as active substance. As far as the assembling in the yeast cells is concerned, the problem of the precipitation of the formed fusion proteins does not exist. The reason for this is presumably that the concentration of the free fusion proteins, i. e. those which are not yet bound to the capsomers, remains always low.

It is of advantage when the third amino acid sequence forms the N end terminal of the fusion protein. This permits with a particularly high probability a correct folding of the active substance presumably because the folding of the active substance, on account of the synthesis taking place from the N end terminal towards the C end terminal, is not influenced by a binding to the capsomers that has already taken place. This results in a particularly effective assembling in the yeast cells.

Preferably, in the co-expression of the proteins and of the fusion proteins the respective extent of the expression of the proteins and of the fusion proteins is harmonised in such a manner that the greatest amount of virus-type particles containing the active substance that is possible in the course of the method is formed. This can be achieved by integrating expression plasmids which code for the proteins and the fusion proteins in a harmonised number of copies into the yeast cells. Nucleic acid sequences, which code for the proteins and the fusion proteins can also be integrated in a harmonised number of copies and in a stable manner into the genom of the yeast cells. In a preferred configuration of the method according to the invention the expression of the proteins is carried out under the control of a first promotor which is contained in a first plasmid coding for the proteins, and the expression of the fusion proteins is carried out under the control of a second promotor which is contained in a second plasmid coding for the fusion proteins. Here, the respective extent of the expression of the proteins and of the fusion proteins is harmonised by a suitable selection of the first and of the second promotor. By means of an appropriate choice of the stoichiometric ratio between the expression of the fusion protein and of the first amino acid sequence or of the protein it is possible to prevent that the fusion protein, the first amino acid sequence or the protein are expressed in the yeast cells in an superfluous amount. When a too big amount of the fusion proteins is expressed, part of the formed fusion proteins is not integrated into VLPs. When a too big amount of the first amino acid sequence or of the protein is expressed, VLPs are formed which do not contain any active substance or only a small amount thereof.

Preferably the first and/or the second promotor are/is selected from a group, consisting of the promoters of the genes of alcohol dehydrogenase 1 (ADH1), alcohol dehydrogenase 2 (ADH2), orthophosphoric monoester phosphohydrolase (Apase), format dehydrogenase (FOD), galactokinase (GAL1), UDP glucose-4-epimerase (GAL10), glyceraldehyd-3-phosphate (GAP), glyceraldehyd-phosphate dehydrogenase (GAPDH), alcohol oxidase (AOX), methanol oxidase (MOX), no message in thiamine 1 (NMT1), 3-phosphoglycerate-kinase (PGK) and pyruvatekinase (PYK1) as well as the hybrid promotors GAL10/PYK1 and ADH2/GAPDH.

The third amino acid sequence preferably comprises a sequence of at least one antigen, of at least one epitope of this antigen or of different epitopes of this antigen. When the third amino acid sequence comprises different epitopes of the antigen, the fusion protein is a so-called multi-epitope construct. This antigen may be a tumor-associated antigen. An antigen is understood to be each protein or peptide which may induce in a mammal or in a human being an immuno-reaction, in particular the formation of cytotoxic T cells. For inducing that immuno-reaction it may be necessary to present the antigen to the immuno-system in a suitable way. A tumor-associated antigen is understood to be an antigen which is expressed by tumor cells in a different way than by the respective not degenerated cells of the same type or an antigen which influences in a specific manner the growth and/or the proliferation of tumor cells. The tumor-associated antigen is preferably selected from the group comprising NY-ESO-I, telomerase reverse transcriptase (TERT), p53, MDM2, CYP1B1, HER-2/new, CEACAM (carcinoembryonic antigen-related cell adhesion molecule 5) and the apoptosis-inhibiting protein Survivin.

In a preferred configuration of the invention, the antigen is an antigen of a pathogen of a viral disease or of an infectious disease. An antigen of a pathogen is an antigen which the pathogen itself contains or for which the pathogen has a coding nucleotide sequence. The viral disease or the infectious disease may be a viral disease with a chronic course or an infectious disease. The antigen may be selected from a group comprising: HIV-associated antigen, HCV-associated antigen, tuberculosis-associated antigen, in particular Ag85A, Ag85B, Rv3407, Esat-6 and Hsp65, malaria-associated antigen, in particular CSP-1, LSA-1, LSA-3 and EXP-1, an antigen associated with a merozoite stage of the malarial parasite, in particular MSP-1, and bilharziosis-associated antigen. The antigen is associated with one of the pathogens when the pathogen expresses the antigen itself or induces its expression in the affected organism.

For inducing an immuno-reaction, it is particularly advantageous when the third amino acid sequence forms an MHC Class I-specific antigen.

In an embodiment of the invention, the first amino acid sequence which is derived from the virus protein 1 of the polyoma virus (VP1) does not have the DNA-binding domain which is contained in the VP1 and/or does not have the nucleic localisation sequence (NLS) which is contained in the VP1. The DNA-binding domain is contained in the NLS or overlaps with the NLS. The function of the NLS is normally the translocation of the VP1 into the cell nucleus. The DNA-binding domain can bind any DNA. By omitting the DNA-binding domain or the NLS, it can be accomplished that little or no undesired DNA is packed from the host organism into the VLPs. This makes it possible to achieve a higher quality of the VLPs. Undesired side-effects by DNA from the host organism are avoided. The tolerance of the VLPs is improved when administered to a mammal.

The yeast cells which are used for co-expression are preferably yeast cells of the species Saccharomyces cerevisiae, Schizosaccharomyces pombe, Pichia pastoris, Hansenula polymorpha, Kluyveromyces lactis or Kluyveromyces marxianus.

The invention relates furthermore to virus-type particles containing proteins each having a first amino acid sequence derived from a first virus protein, and fusion proteins. The first amino acid sequence is an amino acid sequence which is adequate for the formation of capsoid-forming capsomers which specifically bind a second virus protein. Each of the fusion proteins has a second amino acid sequence derived from the second virus protein and specifically binding to one of the capsomers each and a third amino acid sequence which is predominantly hydrophobic and forms an active substance. It has so far not been possible to produce such particles with a predominantly hydrophobic third amino acid sequence. Their production is, however, possible when the proteins and the fusion proteins are co-expressed in yeast cells and the particles are formed in the yeast cells. Another method for their production is not known. Advantageous embodiments of the particles according to the invention follow from the above statements relating to the method according to the invention.

The invention relates furthermore to the use of the particles of the invention as a medicament.

The invention is explained in greater detail by reference to the following exemplary embodiments wherein:

FIG. 1 shows a schematic representation of the virus proteins VP2 and VP3 of the polyoma virus as well as of the VP1-binding domain contained therein,

FIG. 2 shows a Coomassie-stained SDS-polyacrylamide-gel in which samples from fractions 1 to 38 of a caesium-chloride gradient were separated by gel electrophoresis,

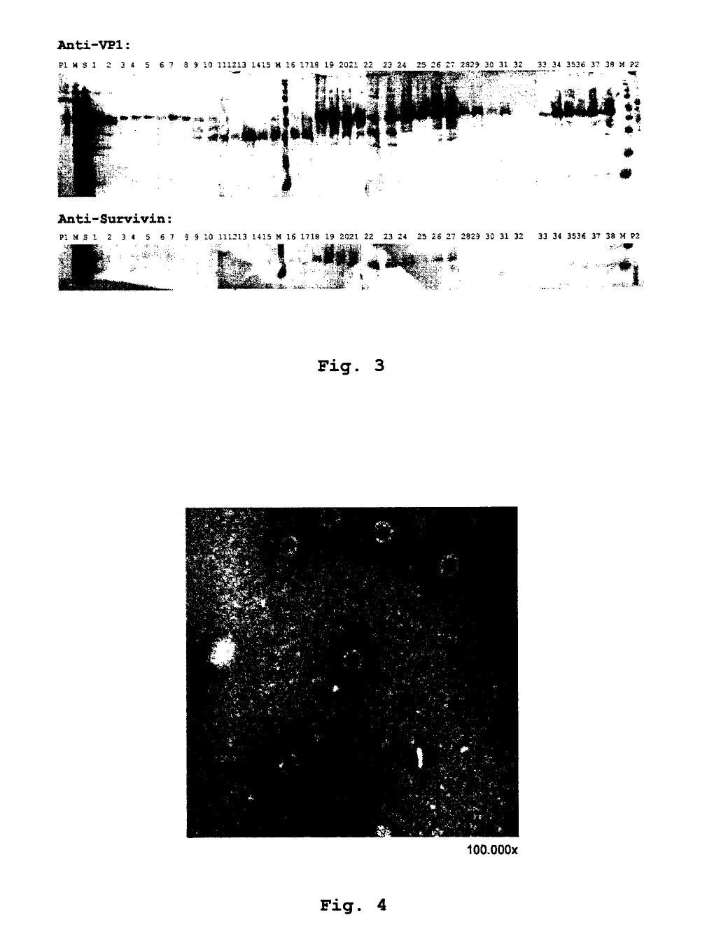

FIG. 3 shows a Western blot analysis with VP1-specific antibodies (above) and Survivin-specific antibodies (below) of the fractions 1 to 38 of the caesium-chloride gradient and

FIG. 4 shows an image made by an electron microscope of the united VP1 peak fractions 24 to 27 of the caesium-chloride gradient, 100,000-fold enlarged.

### EXAMPLE 1

### Production of Virus-Type Particles By Co-Expression of the Virus Protein VP1 of the Polyoma Virus And of A Fusion Protein From The Virus Protein VP3 of the Polyoma Virus And of the Tumor Antigen Her2/New

Three yeast expression plasmids are produced.

The yeast expression plasmid pGCH-VP1 contains
the yeast-specific GAL1 promotor,
a yeast-specific CYC1 transcription termination sequence
a CEN/ARS DNA fragment (origin of replication),
a HIS3 selection marker and
the coding DNA for the polyoma virus protein VP1.

The extracellular domain and the transmembrane domain of Her/2 new (amino acids 1-683) undergoes a translational fusion with VP3. For this, the yeast expression plasmid pGCL-Her2/new (1-683)-VP3 contains
the yeast-specific GAL1 promotor,
a yeast-specific CYC1 transcription termination sequence
a CEN/ARS DNA fragment (origin of replication),
a LEU2 selection marker and
the coding DNA for the fusion protein of Her2/new and VP3, with Her2/new (amino acids 1-683) having undergone a fusion to the N end terminal of VP3 (amino acids 116-297, see FIG. 1).

The yeast expression plasmid pGCL-VP3-Her2/new (1-683) contains
the yeast-specific GAL1 promotor,
a yeast-specific CYC1 transcription termination sequence
a CEN/ARS DNA fragment (origin of replication),
a LEU2 selection marker and
the coding DNA for the fusion protein of VP3 and Her2/new, with Her2/new (amino acids 1-683) having undergone a fusion to the C end terminal of VP3 (amino acids 116-297, see FIG. 1).

For the co-expression of virus-type particles, yeast cells of the strain Saccharomyces cerevisiae (JD53 (leu2, his3, trp1, lys2, ura3) are each transformed with the yeast expression plasmid pGCH-VP1 as well as with the yeast expression plasmid pGCL-Her2/new (1-683)-VP3 or the yeast expression plasmid pGCL-VP3-Her2/new (1-683) according to the method of Schiestl, R. H. and Gietz, R. D. (1989) Current Genetics, Volume 16, pages 339 to 346. The yeast cells transformed with both plasmids are cultured on agar plates with a synthetic SD medium (6.7 g/l YNB (Becton Dickinson GmbH, Heidelberg, Germany), 200 mg/l Lysine, 200 mg/l Tryptophan, 200 mg/l Uracil, 2% glucose) at 30° C. For the production and cleaning of virus-type particles, the transformed cells are cultured in 1,000 ml SD medium. The cultures are cultured up to an optimum density (OD₆₀₀) of 4-8. For the induction of the GAL1 promotors, the yeast cells are subsequently centrifuged at 1,000×g for 2 minutes, and the cell pellets are adjusted by means of SG medium (6.7 g/l YNB (Becton Dickinson GmbH, Heidelberg, Germany), 200 mg/l Lysine, 200 mg/l Tryptophan, 200 mg/l Uracil, 2% galactose) to an OD₆₀₀ of 2 and cultured at 30° C. for another 24 hours.

Thereafter, the yeasts are centrifuged for 10 minutes at 1,000×g, and the yeast pellet is taken up in 2.5 ml cell disruption buffer (20 mM Tris-HCl, pH 7.6, 100 mM NaCl, 1 mM EDTA, 0.1% Triton X-100, 1 mM PMSF) per gram fresh weight of the yeasts. The cell disruption is performed by means of a BeadBeater (Biospec Products, Inc., Bartles-ville, Okla. 74005, USA) using glass beads with a diameter of 0.5 mm. For this purpose, the cell suspension is treated with a BeadBeater under continuous cooling 15 times in intervals of 15 seconds. Thereafter, insoluble residues are separated by means of a centrifuging of 20 minutes at 10,000×g. For the further cleaning of the virus-type particles, 6 ml each of the supernatant are cautiously piled on 32 ml of a saccharose pad (45% saccharose, in cell disruption buffer) and are centrifuged over four hours at 4° C. and 100,000×g. The resulting pellets contain the virus-type particles. The pellets are taken up in cell disruption buffer (without PMSF), and insoluble material is removed by centrifuging at 5,000×g over 10 minutes. The supernatant is applied to a caesium-chloride step gradient. The caesium-chloride step gradient is piled up from 4 ml fractions of increasing density (1.23 g/cm³, 1.26 g/cm³, 1.26 g/cm³, 1.32 g/cm³, 1.35 g/cm³, 1.38 g/cm³ in 20 mM Tris-HCl, pH 7.6. A subsequent centrifuging is performed over 36 hours at 100,000×g and 4° C. Thereafter, 1 ml fractions of the gradient are analysed by means of SDS-polyacrylamide gel electrophoresis and Western blot. The presence of cleaned virus-type particles VP1/VP3-Her2/new (1-683) and/or VP1/Her2/new (1-683)-VP3 is proven by negative contrasting with 1% uranyl acetate in the electron microscope.

### EXAMPLE 2

### Production of Virus-Type Particles By Co-Expression of the Virus Protein VP1 of the Polyoma Virus And of A Fusion Protein From the Virus Protein VP2 of the Polyoma Virus And the Murine Tumor Antigen mSurvivin

The virus protein VP2 of the polyoma virus is known from Abbing, A. et al., 2004, Journal of Biological Chemistry, Volume 279, pages 27410 to 27421.

Two yeast expression plasmids are produced. The yeast expression plasmid pGCH-VP1 has already been described in Example 1.

The fusion of mSurvivin to the N end terminal of VP2 was performed. The yeast expression plasmid pmSurv-VP2 contains
a yeast-specific MET25 promotor, the coding DNA for the fusion protein of mSurvivin (amino acids 1-140) and VP2 (amino acids 252-297,see FIG. 1) followed by a yeast-specific PGK transcription termination sequence
a yeast-specific ADH1 promotor, the coding DNA for the fusion protein of mSurvivin (amino acids 1-140) and VP2 (amino acids 252-297, see FIG. 1) followed by a yeast-specific ADH1 transcription termination sequence,
a 2μ (2 micron) DNA fragment of the yeast 2μ plasmid (origin of replication) and
a TRP1 selection marker.

For the co-expression of virus-type particles, yeast cells of the strain Saccharomyces cerevisiae (JD53 (leu2, his3, trp1, lys2, ura3) are transformed with the two yeast expression plasmids according to the method of Schiestl, R. H. and Gietz, R. D. (1989) Current Genetics, Volume 16, pages 339 to 346.

The yeast cells transformed with both plasmids are cultured as described in example 1. Thereafter, the yeasts are centrifuged for 10 minutes at 1,000×g, and the yeast pellet is taken up in 0.5 ml cell disruption buffer (20 mM Tris-HCl, pH 7.6, 100 mM NaCl, 1 mM EDTA, 0.01% Triton X-100, 1 mM PMSF) per gram fresh weight of the yeasts. The cell disruption is performed by means of a hydraulic press according to the principle of the “French Press” (One Shot, Constant Cell Disruption Systems Ltd., Northants, NN11, 4SD, Great Britain) in three cycles at 2,000 bar. The further treatment of the disrupted cells for cleaning the VLPs expressed therein is performed as described in example 1.

FIG. 2 shows a Coomassie staining of a SDS polyacrylamide gel on which a sample each of the fractions 1 to 38 of the caesium-chloride gradient has been applied by means of gel electrophoresis. On the tracks designated by M, molecular weight markers are applied. On the track designated by P, cleaned VP1 was applied as a positive control. In the fractions 24 to 27, cleaned VP1 can be seen.

FIG. 3 shows a Western blot analysis of the fractions 1 to 38 of the caesium-chloride gradient. In the figure at the top, a Western blot analysis performed with VP1-specific antibodies is shown, and in the figure at the bottom, a Western blot analysis performed with Survivin-specific antibodies is shown. In the fractions 18 to 27, both VP1 and Survivin-VP2-fusion protein can be seen.

The presence of cleaned virus-type particles VP1/mSurvivin-VP2 is proven by negative contrasting with 1% uranyl acetate in the electron microscope. FIG. 4 (enlarged 100,000-fold) shows an electron microscope image of the VLPs which are contained in the united fractions 24 to 27 of the caesium-chloride gradient.

### EXAMPLE 3

As an alternative to the cleaning of the VLPs by means of ultra-centrifugation as described in the examples 1 and 2, the VLPs are cleaned with classical bio-chemical methods after the culturing of the yeasts. For this purpose, the cell pellets are re-suspended in buffer QSA (20 mM ethanolamine, 2 mM EDTA; 6 mM DTT, 50 mM NaCl, 5% glycerine, pH 9.0; 10 ml buffer per gram cell pellet+protease inhibitor+benzonase, final concentration 1 U/ml). The cell disruption is done with a French Press (3 cycles at 2,000 bar). Thereafter, cellular debris is removed by means of centrifugation at 75,000×g over 45 minutes and at 4° C. The pH value of the supernatant is adjusted to 9.0 by the addition of 0.1 M NaOH. The first cleaning is made via cation exchange chromatography (POROS(R) 50 HS, Applied Biosystems, Foster City, Calif. 94404, USA) with a gradient on 100% buffer QSB (20 mM ethanolamine, 2 mM EDTA, 6 mM DTT; ! M NaCl, 5% glycerine, pH 9.0) over 10 column volumes. Fractions with a high VP1 concentration are united, and the conductivity is adjusted to 9 ms/cm through the addition of buffer QSA. This is followed by a cleaning via anion exchange chromatography (Q sepharose (R), GE Healthcare, 80807 Munich, Germany) with a gradient on 60% buffer QSB over 8 column volumes. Once again fractions with a high VP1 concentration are united. The final step is a re-buffering and a further cleaning by means of gel filtration (Superdex (R) 200, GE Healthcare) in PBS buffer. The presence of fractions with cleaned virus-type particles is proven by negative contrasting with 1% uranyl acetate in the electron microscope.

1. A method for producing virus-type particles containing an active substance, proteins each having a first amino acid sequence which is derived from a first virus protein and fusion proteins assemble to the virus-type particles, the first amino acid sequence being an amino acid sequence which is adequate for the formation of capsoid-forming capsomers that specifically bind a second virus protein, the fusion proteins having a second amino acid sequence each derived from the second virus protein and specifically binding to one of the capsomers each and a third amino acid sequence which forms the active substance, the proteins and the fusion proteins being co-expressed in yeast cells.

2. A method according to claim 1, wherein the first virus protein and/or the second virus protein stems/stem from a virus or can be obtained from the same, the virus being selected from the group of the non-enveloped viruses, comprising Papovaviridae, in particular Polyoma and Papilloma viruses, Iridoviridae, Adenoviridae, Parvoviridae, Picornaviridae, in particular polio viruses, Caliciviridae, Reoviridae and Birnaviridae.

3. A method according to any of the preceding claims, the first virus protein being the virus protein 1 of the polyoma virus (VP1) and the second virus protein being the virus protein 2 (VP2) or the virus protein 3 (VP3) of the polyoma virus.

4. A method according to any of the preceding claims, the capsomers having the form of pentamers, hexamers or heptamers.

5. A method according to any of the preceding claims, the third amino acid sequence being, at least predominantly, hydrophobic.

6. A method according to any of the preceding claims, the third amino acid sequence forming the N terminal end of the fusion protein.

7. A method according to any of the preceding claims, wherein in the course of the co-expression of the proteins and of the fusion proteins the respective extent of the expression of the proteins and of the fusion proteins is harmonised in such a way that in the course of the method the biggest possible amount of the virus-type particles containing the active substance is formed.

8. A method according to claim 7, the expression of the proteins taking place under the control of a first promotor contained in a first plasmid, which is coding for the proteins and the expression of the fusion proteins taking place under the control of a second promotor contained in a plasmid, which is coding for the fusion proteins, the respective extent of the expression of the proteins and of the fusion proteins being harmonised by means of a suitable selection of the first and of the second promotor.

9. A method according to claim 8, the first and/or the second promotor being selected from a group consisting of the promotors of the genes of alcohol dehydrogenase 1 (ADH1), alcohol dehydrogenase 2 (ADH2), orthophosphoric monoester phosphohydrolase (Apase), format dehydrogenase (FOD), galactokinase (GAL1), UDP glucose-4-epimerase (GAL10), glyceraldehyd-3-phosphate (GAP), glyceraldehyd-phosphate dehydrogenase (GAPDH), alcohol oxidase (AOX), methanol oxidase (MOX), no message in thiamine 1 (NMT1), 3-phosphoglycerate-kinase (PGK) and pyruvatekinase (PYK1) as well as the hybrid promotors GAL10/PYK1 and ADH2/GAPDH.

10. A method according to any of the preceding claims, the third amino acid sequence comprising a sequence of at least one antigen, in particular a tumor-associated antigen, of at least one epitope of this antigen or of different epitopes of the said antigen.

11. A method according to claim 10, the tumor-associated antigen being selected from the group comprising NY-ESO-I, Telomerase Reverse Transcriptase (TERT), p53, MDM2, CYP1B1, HER-2/new, CEACAM (carcinoembryonic antigen-related cell adhesion molecule 5) and the apoptosis inhibiting protein Survivin.

12. A method according to any of the claims 1 to 10, the antigen being an antigen of a pathogen of a virus disease, in particular, a chronic one, or of an infectious disease.

13. A method according to claim 12, the antigen being selected from a group comprising HIV-associated antigen, HCV-associated antigen, tuberculosis-associated antigen, in particular Ag85A, Ag85B, Rv3407, Esat-6 and Hsp65, malaria-associated antigen, in particular CSP-1, LSA-1, LSA-3 and EXP-1, an antigen associated with a merozoite stage of the malarial parasite, in particular MSP-1, and bilharziosis-associated antigen.

14. A method according to any of the preceding claims, the third amino acid sequence forming an MHC-Class I-specific antigen.

15. A method according to any of the claims 3 to 14, wherein the first amino acid sequence which is derived from the virus protein 1 of the polyoma virus (VP1) does not have the DNA-binding domain contained in the VP1 and/or does not have the nuclear localisation sequence (NLS) contained in the VP1.

16. A method according to any of the preceding claims, the yeast cells being yeast cells of the species Saccharomyces cerevisiae, Schizosaccharomyces pombe, Pichia pastoris, Hansenula polymorpha, Kluyveromyces lactis or Kluyveromyces marxianus.

17. Virus-type particles containing proteins each having a first amino acid sequence which is derived from a first virus protein, and fusion proteins, the first amino acid sequence being an amino acid sequence which is adequate for the formation of capsoid-forming capsomers which specifically bind a second virus protein, the fusion proteins having a second amino acid sequence each which is derived from the second virus protein and specifically binds to one of the capsomers each and a predominantly hydrophobic third amino acid sequence which forms an active substance.

18. Particles according to claim 17, wherein the first virus protein and/or the second virus protein stems/stem from a virus or can be obtained from the same, that is selected from the group of the non-enveloped viruses, comprising Papovaviridae, in particular Polyoma and Papilloma viruses, Iridoviridae, Adenoviridae, Parvoviridae, Picornaviridae, in particular polio viruses, Caliciviridae, Reoviridae and Birnaviridae.

19. Particles according to any of the claims 17 or 18, the first virus protein being the virus protein 1 of the polyoma virus (VP1) and the second virus protein being the virus protein 2 (VP2) or the virus protein 3 (VP3) of the polyoma virus.

20. Particles according to any of the claims 17 to 19, the capsomers having the form of pentamers, hexamers or heptamers.

21. Particles according to any of the claims 17 to 20, the third amino acid sequence forming the N terminal end of the fusion protein.

22. Particles according to any of the claims 17 to 21, the third amino acid sequence comprising a sequence of at least one antigen, in particular a tumor-associated antigen, of at least one epitope of this antigen or of different epitopes of the said antigen.

23. Particles according to claim 22, the tumor-associated antigen being selected from the group comprising NY-ESO-I, Telomerase Reverse Transcriptase (TERT), p53, MDM2, CYP1B1, HER-2/new, CEACAM (carcinoembryonic antigen-related cell adhesion molecule 5) and the apoptosis inhibiting protein Survivin.

24. Particles according to any of the claims 17 to 22, the antigen being an antigen of a pathogen of a virus disease, in particular, a chronic one, or of an infectious disease.

25. Particles according to claim 24, the antigen being selected from a group comprising HIV-associated antigen, HCV-associated antigen, tuberculosis-associated antigen, in particular Ag85A, Ag85B, Rv3407, Esat-6 and Hsp65, malaria-associated antigen, in particular CSP-1, LSA-1, LSA-3 and EXP-1, an antigen associated with a merozoite stage of the malarial parasite, in particular MSP-1, and bilharziosis-associated antigen.

26. Particles according to any of the claims 17 to 25, the third amino acid sequence forming an MHC-Class I-specific antigen.

27. Particles according to any of the claims 19 to 26, where the first amino acid sequence which is derived from the virus protein 1 of the polyoma virus does not have the DNA-binding domain contained in the virus protein 1 and/or does not have the nuclear localisation sequence (NLS) contained in the virus protein 1.

28. Particles according to any of the claims 17 to 27 for use as a medicament.
